(19) United States
(12) Patent Application Publication
(10) Pub. No.: US 2013/0276365 A1
(43) Pub. Date: Oct. 24, 2013

(54) LIGHT GUIDE DEVICE AND APPARATUS FOR TRANSMITTING LIGHT INTO A CULTURE SOLUTION

(71) Applicant: James Bruce Franklin, Ultimo (AU)

(72) Inventor: James Bruce Franklin, Ultimo (AU)

(73) Assignee: University of Technology, Sydney, New South Wales (AU)

(21) Appl. No.: 13/991,385

(22) PCT Filed: Nov. 30, 2011
(86) PCT No.: PCT/AU11/01560

§ 371 Date: Jun. 27, 2013

(30) Foreign Application Priority Data
Dec. 3, 2010 (AU) 2010905323

(51) Int. Cl. C12M 1/00; G02B 19/00; G02B 27/00

(52) U.S. Cl. CPC C12M 21/02; G02B 27/00; G02B 19/0019
USPC 47/1.4; 359/894; 359/857

(57) ABSTRACT

A light guide device for transmitting light through a column of light transmitting medium and into an adjacent culture growing solution, including a barrier structure for separating the medium and solution, the barrier structure being light transmissive to allow for extraction of light and being adapted to hold a material of reduced refractive index relative to the medium so as to provide total internal reflection for light incident the barrier structure at greater than a critical angle to thereby guide the light through the device.

### TECHNICAL FIELD

The invention generally relates to light guides for distributing light into a culture solution in which photosynthetic organisms such as algae may be grown.

### BACKGROUND

The growth of algae is influenced by the intensity and wavelength of incident light. In situations where algae are cultivated outdoors, in ponds or the like, the algae may be exposed to direct sunlight. In direct sunlight algae can become photo-inhibited and the growth rate is reduced.

Additionally, algae nearer the surface can shade algae growing below which also reduces the growth rate of the algae lower in the water.

As such, various systems have been proposed to control the amount of light entering the water in an attempt to influence the growth rate of algae. In particular, these systems typically utilise a light guide to distribute light entering the water column more evenly over depth into an associated photosynthetic culture.

A known form of light guide is made of solid plastics material, which needs to be of high quality for efficient transmission of the light. However, costs associated with the production of such materials render the light guide unsuitable for large scale projects.

### SUMMARY

In accordance with a first aspect there is provided, a light guide device for transmitting light through a column of light transmitting medium and into an adjacent culture growing solution, the light guide device including a barrier structure for separating the medium and solution, the barrier structure being light transmissive to allow for extraction of light and being adapted to hold a material of reduced refractive index relative to the medium so as to provide total internal reflection for light incident the barrier structure at greater than a critical angle to thereby guide the light through the device.

Preferably, the barrier structure includes a first wall for containing the medium and a second wall separated from the first wall by a gap for containing the material.

Preferably, the medium is in the form of a column of liquid, and the material is a gas.

In another aspect there is provided, a light guide device for transmitting light including a barrier structure with first and second walls for separating a column of light transmitting medium and a culture solution; wherein the first and second walls are separated by a gap; and wherein the first and second walls are light transmissive to allow light to be extracted from the medium, into the culture solution.

Preferably, the medium is a liquid.

Preferably, the gap is filled with a gas to allow light incident on the barrier structure, within the column of liquid, to be transmitted through the column of liquid by total internal reflection.

Preferably, the gas is air.

Preferably, the first and second walls are composed of polymer sheets.

Preferably, portions of the first and second walls are joined so as to form joined portions.

Preferably, the gap is formed of multiple gap portions defined between joined portions of the walls.

Preferably, the minimum width of the gap is 1 micrometer.

In one form, the device includes a wavelength shifting element for shifting the wavelength of light incident the device.

In one form, at least one of the first and second walls includes the wavelength shifting element.

In one form, the wavelength shifting element is provided in the form of a sheet material located within the culture solution.

In one form, the wavelength shifting element includes fluorescent or phosphorescent materials.

Preferably, the device is in the form of a cell and the barrier structure is configured to contain either the medium or the culture solution, separated from the adjacent other of the medium or the culture solution.

In yet another aspect there is provided, a culture apparatus, including an array of cells formed in accordance with the cell as defined above.

Preferably, the adjacent cells share a common barrier structure.

Preferably, covers are provided over the culture solution to protect the culture from direct light.

Preferably, the covers are configured to direct light from over the culture solution into the light guide device.

In still yet another aspect, there is provided a culture apparatus including a cell formed in accordance with the cell as defined above, wherein the cell has opposing side walls including the barrier structure, the side walls diverging from one another from a first end of the cell towards a second end of the cell.

Preferably, there are at least two said cells within the medium, the cells being arranged side-by-side with a column of the medium therebetween, the first end of each cell being located towards a surface of the medium and the second end of each cell being located away from the surface of the medium.

Preferably, the or each cell is substantially triangular in shape when viewed from one of the front end and the back end of the cell.

Preferably, the or each cell contains the culture solution and the medium is water.

In still yet another aspect, there is provided a method of transmitting light through a device and into the culture solution, including: guiding light through a column of liquid, away from a light receiving end of the device, by total internal reflection from a gas provided adjacent the column of liquid; and extracting the light from the column of liquid.

### BRIEF DESCRIPTIONS OF THE FIGURES

The invention is described, by way of non-limiting example only, by reference to the accompanying figures, in which;

FIG. 1 is a segmented view of two adjacent light guide devices;

FIG. 2 is a side view of first and second walls of the light guide device;

FIG. 3 is a side view of a gap portion between the first and second walls, illustrating total internal reflection;

FIGS. 4a, 4b, 4c and 4d are ray tracing diagrams;

FIG. 5 is a side view of the light guide device in the form of a device immersed in a vessel containing the culture' solution;

FIG. 6 is a side view of a cell immersed in a vessel containing the culture solution with light scattering particles added to the column of water;

FIG. 7 is a side view of the first and second walls of the cells including wavelength shifting elements.

FIG. 8 is a side view of a culture apparatus suitable for outdoor cultivation of algae;

FIG. 9 is a top view of the culture apparatus with light receiving ends of the devices and covers of associated algae cells;

FIG. 10 is a top view of the culture apparatus where the light receiving ends of the devices s are circular;

FIG. 11 is a side view of a culture apparatus suitable for outdoor cultivation of algae;

FIG. 12 is a view of another device with a barrier separating a first and a second fluid;

FIGS. 13a, 13b and 13c illustrate three alternative arrangements of the device;

FIG. 14a illustrates a perspective sectional view of an algae cell for use in a culture apparatus for cultivation of algae;

FIG. 14b illustrates a sectional view of the culture apparatus with a plurality of the algae cells within a water medium; and

FIG. 14c illustrates a detailed view of the barrier structure of the algae cell.

### DETAILED DESCRIPTION

FIG. 1 illustrates a light guide device 1 for transmitting light through a column of light transmitting medium 3a, which is in the form of a column of water 3, into an adjacent culture solution 5. The device 1 including a barrier structure 6 for separating the column of water 3 and culture solution 5.

The barrier structure 6 is light transmissive to allow for the extraction of light and being adapted to hold a material (9a), which is preferably a gas such as air 9, of reduced refractive index relative to the water 3 so as to provide total internal reflection for light incident the barrier structure 6 at greater than a critical angle to thereby guide the light through the device 1.

The barrier structure 6 includes a first wall 2 for containing the column of water 3 and an adjacent second wall 4, for separating the device 1 from the surrounding culture solution 5. The first and second walls are separated by a gap 10 and the first 2 and second walls 4 are light transmissive to allow light to be extracted from the column of water 3 into the culture solution 5. The culture solution 5 includes algae in a growth medium that may be predominantly water based.

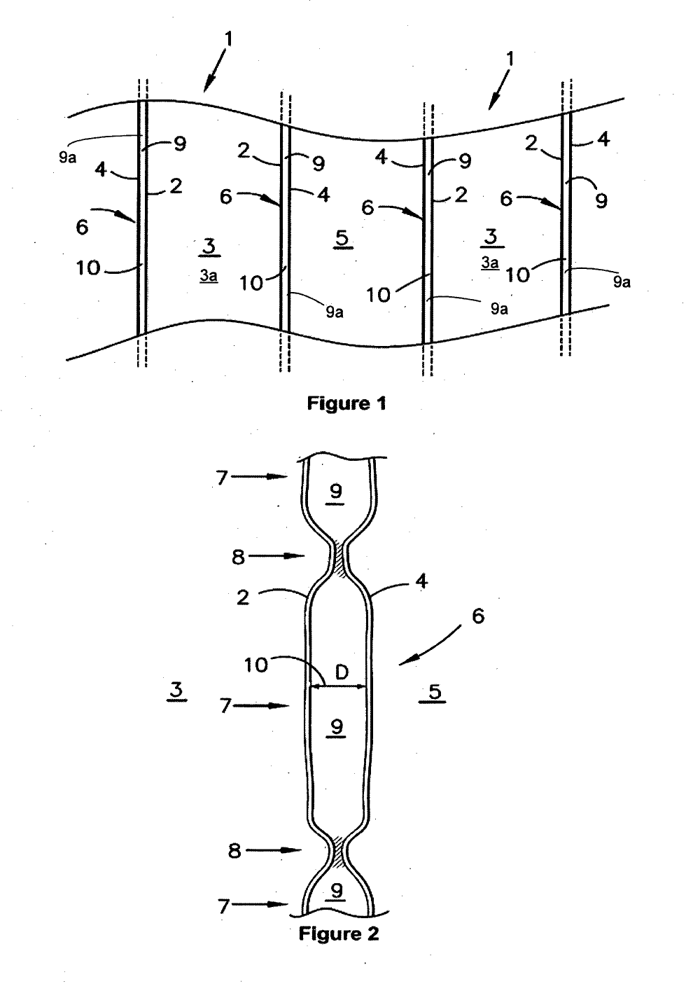

FIG. 2 illustrates the first 2 and second walls 4 as being connected together at joined portions so as to define gap portions therebetween. The gap portions 7 are filled with a gas, which is preferably air 9. Alternatively, the air 9 may be replaced by another gas such as, for example, nitrogen, argon or carbon dioxide. It is envisaged that the use of an inert gas, such as argon, may in some instances be advantageous due to the reduced permeability of the first 2 and second 4 walls. The minimum width, as indicated in FIG. 2 by “D”, of the gap portions 7 is at least the larger of 1 micrometer or two wavelengths of the incident light.

The first 2 and second walls 4 are formed of a light transmissive polymer, such as clear plastic sheets and may be joined such as by heat welding. Joined portions 8 may also be formed in mesh or weave patterns adjacent gap portions 7. In this form, it may then be appreciated that the barrier structure 6 is in form of a transparent gas filled bi-layer barrier structure that provides light transmission between the column of water 3 and the culture solution 5.

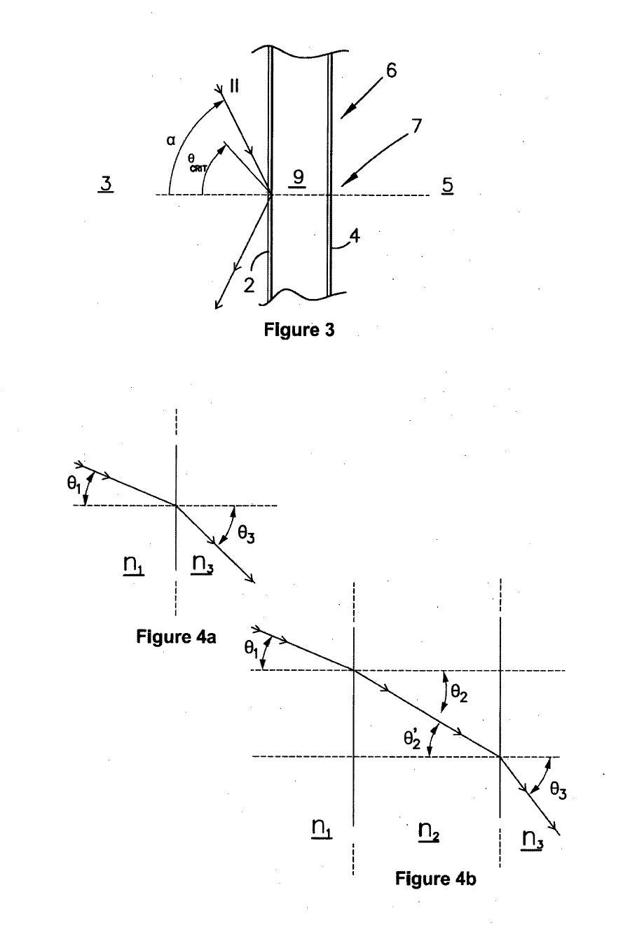

FIG. 3 illustrates a detailed view of the barrier structure 6 with an illustrative light ray 11 incident on the first wall 2 with an incidence angle α normal to the surface of the first wall 2. In this configuration, the light ray 11 will encounter the gas 9 located between the column of water 3 and the culture solution 5. As the gas 9 has a refractive index of approximately 1.0003 and the column of water 3 approximately 1.33, the incident light ray 11 will be totally internally reflected for incident angles which exceed the critical angle θ_{crit} (which in this example is approximately 49 degrees). In this Figure, incidence angle α is shown to be greater than critical angle θ_{crit} hence total internal refection of the light ray 11 will occur.

The presence of the first wall 2 between the column of water 3 and the air 9 does not affect the critical angle and therefore does not affect total internal reflection of the light ray. The reason for this is that the first wall 2 is formed of a polymer which has a higher refractive index than both air and water. A proof of this to demonstrate that the angle for total internal reflection is unchanged, despite the presence of the first wall 2 is given below.

We wish to show that the critical angle for light coming from a medium of refractive index n₁ to a medium of lower refractive index, n₃, is unchanged by interposing a layer of higher refractive index n₂ where n₂>n₁>n₃.

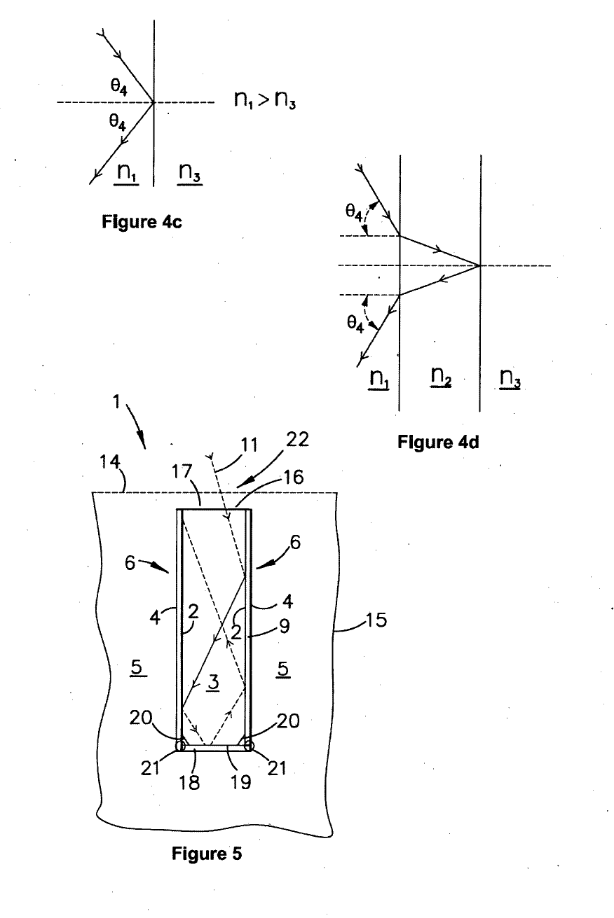

Referring to FIG. 4a illustrating an water-air interface where n₁>n₃, by Snell's law

$$\begin{matrix}{n}_{1} sin {θ}_{1} = {n}_{3} sin {θ}_{3} , & ( 1 ) \\ ∴ {θ}_{crit} = {sin}^{- 1} ( \frac{{n}_{3}}{{n}_{1}} ) . & ( 2 )\end{matrix}$$

Referring to FIG. 4b illustrating a plastic sheet of refractive index n₂ inserted between the water-air interface, where n₂>n₁>n₃, we see by Snell's law at the boundary of n₁ and n₂ that

n₁ sin θ₁=n₂ sin θ₂ (3)

now the sheet is thin and smooth so

θ′²=θ₂ (4)

hence by Snell's law at the boundary of n₂ and n₃

n₂ sin θ′₂=n₃ sin θ₃ (5)

combining (3), (4) & (5),

n₁ sin θ₁=n₂ sin θ₂=n₂ sin θ′₂=n₃ sin θ₃; (6)

∴n₁ sin θ₁=n₃ sin θ₃; (7)

Thus,

$${θ}_{crit} = {sin}^{- 1} ( \frac{{n}_{3}}{{n}_{1}} ) ,$$

(8)
which is the same as (2) and therefore the presence of the intermediate layer of refractive index n₂ does not affect θ_{crit}.

FIG. 4c shows total internal reflection occurring at some angle θ₄≧θ_{crit} at the interface between materials of refractive index n₁ and n₃. As shown in FIG. 4d, interposing a layer of higher refractive index n₂ where n₂>n₁>n₃ shifts the point of total internal reflection from the n₁-n₃ interface to the n₂-n₃ interface, but it does not change the ultimate angle of the reflected ray. (The reflected ray is displaced sideways by a small multiple of the thickness of the intermediate layer, however this layer is thin and so the sideways translation is of no consequence.)

In light of the above, it may be appreciated that other materials may be used instead of water in the column 3 and the gas 9 as long as n₁>n₃. Accordingly, the column of water 3 may be filled with another liquid, such, as glycerine, which is a possible bi-product of the algae growth process. In that case, the gas may be also replaced with water. Given, glycerine has a higher refractive index than water n₁>n₃ will still be satisfied and total internal reflection is still able to occur.

FIG. 5, illustrates a preferred example of the device 1 in the form of a cell 22 where the barrier structure 6 is configured to contain the column of water 3 from the surrounding culture solution 5. Alternatively, the cell 22 may contain the culture solution 5 from a surrounding column of water 3. In either case, the barrier structure 6 separates the column of water 3 from the culture solution 5.

The cell 22 is at least partially immersed in the culture solution 5 contained by a vessel 15 such that the cell 22, hence the column of liquid 3, extends from an upper end 16 near the surface 14 of the culture solution 5 downwardly into the culture solution 5. The vessel 15 may be a small or large tank, a pond, a dam or other water containing structure into which the cell 22 may be immersed.

The barrier structure 6 extends from the upper end 16 substantially downwardly into the vessel 15 to a base 18. The base 18 may be formed substantially similarly to the bi-layer barrier structure 6 or have a reflective surface such as a mirror 19 to reflect light. Alternatively, instead of the mirror 19, a diffuse reflector may be used to reflect the light. However, if a diffuse reflector is used the efficiency will be lower. It is envisaged that the base 18 may further include ballast 20 or attachment points 21 to which restraints may be attached to secure the cell 22 with the vessel and maintain the barrier structure 6 in a preferred vertical orientation. Preferably the orientation is substantially vertical. Alternatively, an inclined orientation may be chosen to better match the seasonal variation sunlight with the optimal light pattern for algal growth.

The cell 22 has a light receiving end 17 at the upper end 16 to convey the light into the cell 22. As illustrated in FIG. 5, for example, the light ray 11 passes into the light receiving end 17 and is totally internally reflected, assuming the angle of incidence is greater than the critical angle as described in FIG. 3, from the air 9 in the barrier structure 6. Note that illustration of the light ray 11 in FIGS. 5, 6 and 7 is illustrative only. For example, the light ray 11 may refract as it enters the surface 14 of the culture solution 5 when passing from an external air filled environment.

Additionally, if the base 18 has a mirror 19 the light will be further reflected off the mirror 18 and again totally internally reflected from barrier structure 6 if the incident angle is greater than the critical angle. Therefore, it may be appreciated that in a preferred form the cell 22 forms a light trapping structure from which the light may then be extracted, using any one of a number of known techniques, into the surrounding culture solution 5.

In one form, it is envisaged that the ratio of the vertical height of the cell 22, from the base 18 to the upper end 16, to the width of the light receiving end 17 will be in the range of, say, 4:1 to 20:1. Preferably, the ratio is 10:1 such that, for example, if the vertical height of the device is 1 m the width of the light receiving end 17 will be 0.1 m. Accordingly, it may be appreciated that the relatively narrow aperture of the light receiving end 17 relative to the vertical height is configured such that incident light is received at angles greater than the critical angle such that total internal reflection occurs.

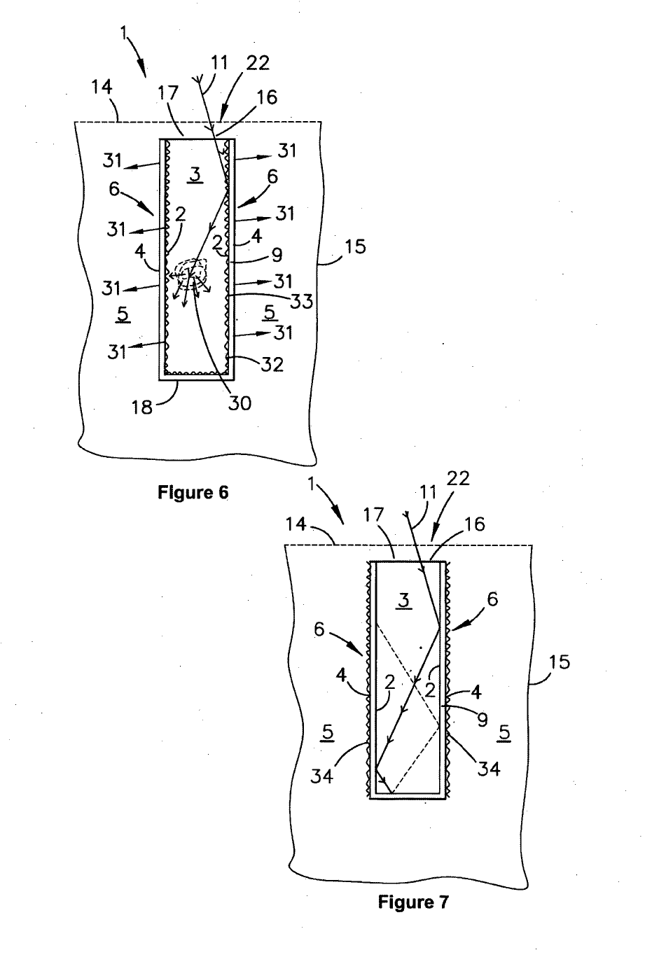

FIG. 6 illustrates an example whereby the incident ray of light 11 passes into the light receiving end 17 and is totally internally reflected, assuming the incident angle is greater than the critical angle as described in FIG. 3, from the barrier structure 6 and is then further reflected from the mirror. As aforementioned, in regard to the example illustrated in FIG. 5, the cell 22 acts as a light trapping structure and the intention is to release the trapped light so it may be distributed into the culture solution 5. Accordingly, the column of liquid 3 may contain light scattering particles 30 to create scattered light 31, as one possible means of extracting light. Preferably, these light scattering particles 30 are in the form of micro-particles configured to minimise backscattering.

As may be appreciated, a portion of the scattered light 31 will have incident angles less than the critical angle and hence a portion of the scattered light 31 will be transmitted into the culture solution 5. Accordingly, as the light ray 11 is totally internally reflected and scattered, the algae are not subject to direct light but rather a more distributed source of light throughout the depth of the culture solution 5. Also, if the base 18 was formed of the barrier structure 6 rather than the mirror, light may also pass through the base 18 into the culture solution 5.

To induce further scattering of, for example the incident light ray 11, the first wall 2 may further include an embedded form of the light scattering particles 30 and/or a light scattering element 32. This light scattering element 32, for example, may include a surface texture 33 onto the first walls 2 such that light is scattered. Preferably, the surface texture 33 is configured such that light is extracted uniformly over the depth of the cell 22. For that purpose, the texture 33 may be graduated to control scattering and provide uniform intensity as light penetrates deeper into the cell 22.

FIG. 7 illustrates the second walls 4 as including a wavelength shifting element 34. The wavelength shifting may be accomplished by fluorescence, phosphorescence or other means. The advantage of using a wavelength shifting element 34 is that the incident sunlight can be wave length shifted to the wavelengths preferred for algal photosynthesis. For example, wavelengths approximately centred on approximately 430 nm and 660 nm are typically preferred.

Preferably, the wavelength-shifting element 34 can be formed by incorporating fluorescent or phosphorescent materials into or on the second wall 4. Alternatively, the wavelengthshifting element 34 can be incorporated into or on the first wall 2. Preferably the wavelength shifting element 34 is a polymer sheet doped with fluorescent or phosphorescent materials. Alternatively, the wavelength shifting element 34 is a polymer sheet doped with fluorescent or phosphorescent materials laminated to one or more clear polymer sheets. An advantage of this embodiment for a second wall 4 is that the surface in contact with the culture solution 5 may be optimized for biocompatibility with the algae without compromising the wavelength shifting performance of the second wall 4. In a further variation, the wavelength shifting element 34 may be provided in the form of one or more sheets of fluorescent or phosphorescent material located within the culture solution adjacent to or in a selected spaced relationship relative to the second wall 4. If the wavelength shifting element 34 is located within the culture solution 5 or, on or in either the inner or outer surfaces of the second wall 4, it is feasible to design the system so that a very high fraction of the wavelength shifted light enters the culture solution. In a further variation, if algae within the culture solution 5 absorb light at a plurality of wavelengths, the wavelength shifting element 34 may contain a plurality of fluorescent or phosphorescent materials that shift the wavelengths of incident light such that the wavelength shifting element 34 emits a plurality of wavelengths to match the wavelengths at which the algae absorb.

However, if the wavelength-shifting element 34 is inside or on the surface of the first wall 2 a significant fraction of the wavelength shifted light may escape up the light transfer cell 22.

Additionally, by shifting the wave length of the light after the light has been transmitted to either the first wall 2 and/or the second wall 4, which are close to where the light is required, the efficiency of the wavelength shifting is potentially improved over systems which perform wavelength shifting then transmit the light to where it is needed.

In yet another from of the invention there is provided, a culture apparatus, including an array of cells formed in accordance with the cell as defined above.

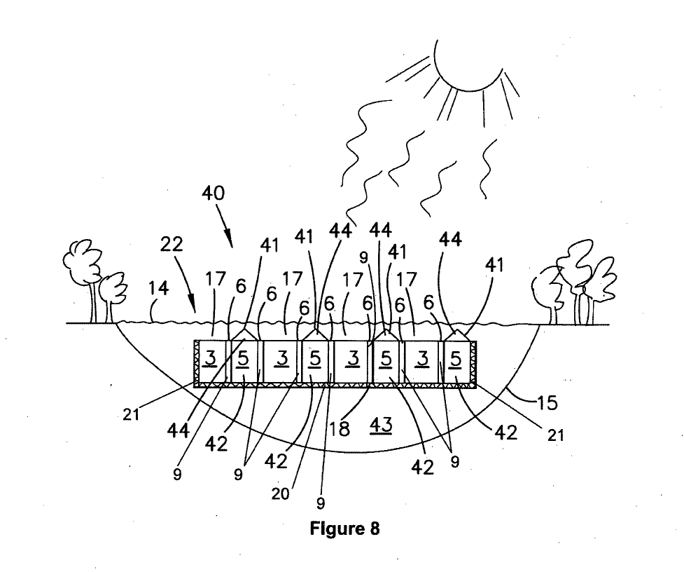

FIG. 8 illustrates a culture apparatus 40 including an array of cells 22, as described above, the cells 22 being spaced apart such that the second walls 2 of the barrier structures 6 define spaces which form algae cells 42 for containing the culture solution 5. Accordingly, the cells 22 and the algae cells 44 share common barrier structures 6.

In this example, the culture apparatus 40 is immersed in a fluid 43 so that at least a thin layer of the fluid 43 overlays the devices 1, to assist in keeping the apparatus 40 clean and protected from the external environment.

The algae cells 42 include opaque covers 41 which at least partially filter any incident light. Importantly, the covers 41 reduces the light intensity near the surface 14 as the light intensity is too high for optimal growth of algae toward the surface 14 of the culture solution 5. The covers 41 also serve to contain the culture solution 5 within the algae cells 42. The covers 41 may include a directing element 44 which is reflective and is shaped so as to direct light incident on the covers 41 into the light transport devices 3 and hence increase the amount of light reaching the culture solution 5. In one form, the directing element 44 may be in the form of a convex surface with the tip submerged below the surface 14. By utilising the directing element 44 to increase the amount of light reaching the culture solution 5, the efficiency of the culture apparatus 40, which is measured in bio-productivity per squire metre, may be increased to potentially double the bio-productivity. It may be appreciated that in one form the covers 41 and directing elements 44 are separate devices, however, in another form the covers 41 and directing elements 44 are the same device such that the directing elements 44 also act as the opaque covers 41.

The covers 41 may also be formed so as to be permeable to allow excess carbon dioxide and/or oxygen to egress from the algae cells 42. In either case, the covers 41 need to be of simple construction for manufacturing purposes and light weight to avoid excess weight load on apparatus 40. In that regard, it is envisaged the covers 41 may be in the form of a membrane which might be supported by gasses within the device to minimise structural components.

In this configuration, the bi-layer barrier structure 6 provides barriers not only between the cells 22 and algae cells 42, but also between the cells 22, the algae cells 42 and the fluid 43. Additionally, the base 18 extends between the bi-layer barrier structure 6 containing the algae cell 42 as well as the column of water 3. As such, the culture apparatus 40 is able to be deployed so as to be submersed within the vessel 15 so that neither the culture solution 5 nor the column of water 3 becomes mixed with the fluid 43.

Additionally, when the devices 1 are used in the apparatus 40, the cells 22 and the algae cells 42, which are essentially water filled, are substantially in hydrostatic equilibrium with the fluid 43. Accordingly, there are relatively small forces on the apparatus system 40 aside from perhaps the buoyancy forces of the air 9 and associated reaction forces from the ballast 20 or attachment points 21. This allows the structure of cultivation system 40 to be composed predominantly of relatively thin bi-layer plastic barrier structures 6.

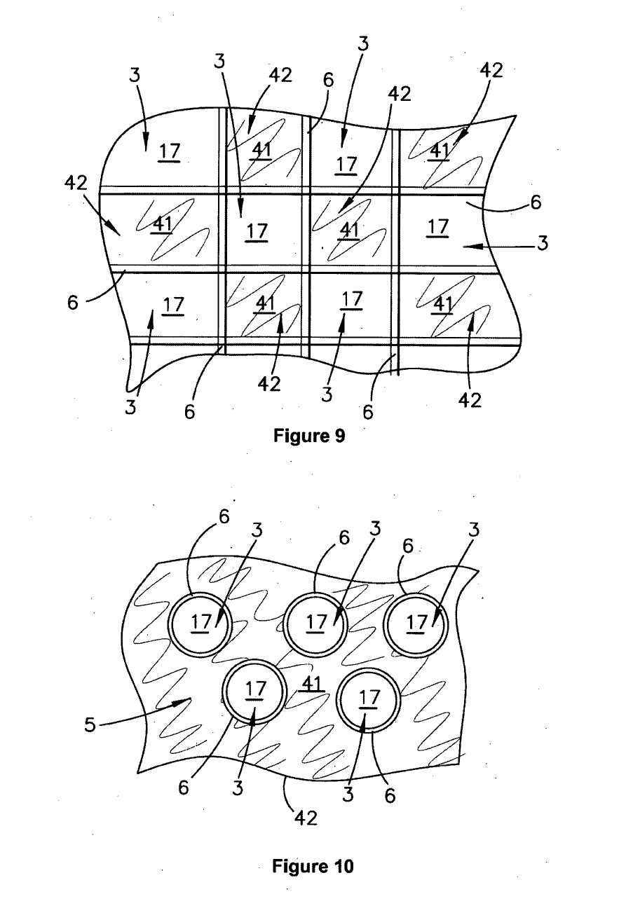

FIG. 9, illustrates an example where the plurality of devices 1 are configured to form a matrix of columns of liquid 3 for distribution of light into adjacent algae cells 42 containing the culture solution 5 below the covers 41.

FIG. 10 shows a further example, wherein the devices 1 of the culture apparatus 40 are circular in shape. In this configuration, the algae cells 42 are irregularly shaped, being defined by the placement of the devices 1, and are immersed in culture solution 5 that is hidden from view below the cover 41. It is also envisaged that this configuration could be reversed, such that the algae cells 42 are circular in shape and surrounded by an irregular shaped cell 22. The common feature of both the above configurations is the barrier structure 6 between the column of water 3 and the culture solution 5.

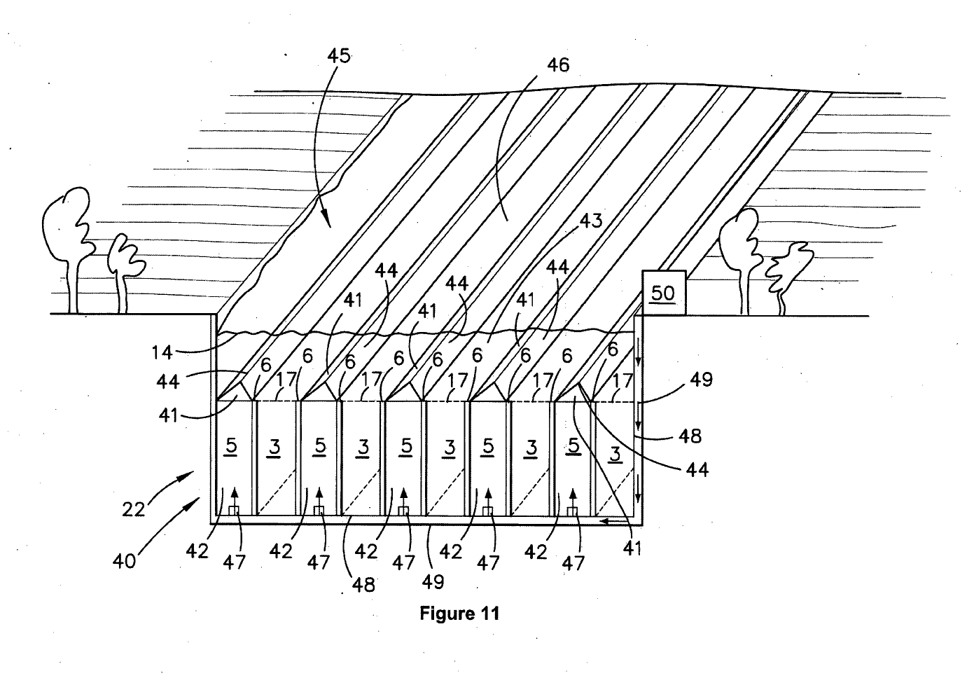

FIG. 11 shows another example, wherein the cells 22 of the culture apparatus 40 are configured so as to be long rectangular prisms 46 anchored to the bottom of a channel 45. Typically, when configured as a long rectangular prisms 46 the light receiving end 17 of the devices 1 may be approximately 100 mm wide and 100 metres long. The algae cells 42 may further include a plurality of ports 47 through which an operation fluid 49 such as carbon dioxide, oxygen and algae growth medium may be inputted or outputted. The ports 47 may then be connected to a corresponding pipe 48 of the channel 45 to transport the operation fluid 49 to and/or from a location 50. Additionally, it may be appreciated that the channel 45 provides structure support against any buoyancy forces associated with the apparatus 40 when configured to be submersed.

Advantageously, the present invention provides a cell 22 for use with culture apparatus 40 in which the principle components are simply the plastic barrier structure 6, the air 9, and the water filled column 3. The cell 22 enables light to be trapped by total internal reflection from the air 9 contained between the first 2 and second 4 walls of the barrier structure 6. The trapped light can then be scattered by various means, such as the aforementioned light scattering particles 30, so that the light is able to pass across the plastic barrier structure 6 and into the culture solution 5. The total internal reflection and light scattering thereby provides control of the intensity and distribution of light as the light passed from the column of water 3 and into the culture solution 5. Furthermore, where wavelength shifting elements 34 are added to the cell 22 the wave length of the light can be shifted to the preferred wavelength for growth of algae, optimising the growth of the algae.

Accordingly, the thin bi-layer barrier structure 6 of the device 1 provides a simple and low cost light guide structure relative to known prior art light guides made of high quality solid plastics material. The device 1, being able to deliver light, over depth, to an associated culture solution 5 with intensity and a wavelength preferred for increased algal growth.

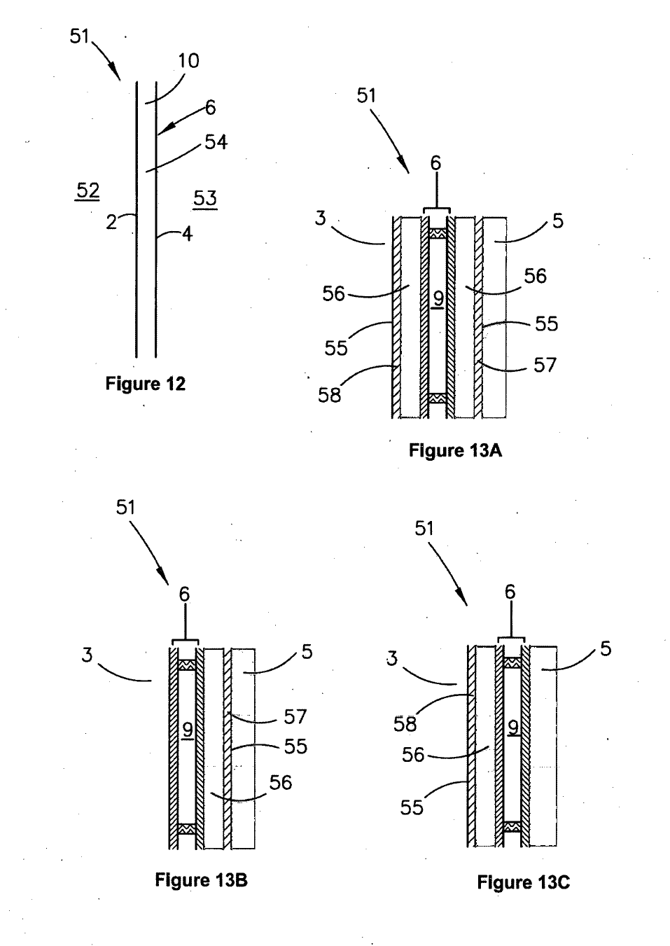

As may be appreciated, the above examples of light guides and cells have been described specifically with reference to the light transmission medium being water and the gas within the barrier being air. However, the principles of the invention are still embodied in the more generalised structure shown in FIG. 12. In this regard, FIG. 12 illustrates a more generic device 51, where the like parts are denoted with like reference numerals. The device 51 includes a barrier structure 6 between a first fluid 52 and a second fluid 53. The barrier structure 6 is light transmissive and includes a third fluid 54 with a lower refractive index than the first fluid 52 such that light in the first fluid 52 which is incident of the barrier structure 6 undergoes total internal refection.

The barrier structure 6 includes a first wall 2 between the first fluid 52 and the third fluid 54 and a second wall 4 between the third fluid 54 and the second fluid 53. Where the first 2 and second walls 4 are separated by a gap 10 in which the third fluid 54 is contained.

In this form, it may be appreciated that the device 51 may be configured to transmit light through to any suitable medium, although, the first fluid 52 is preferably in the form of a column of water 3, the second fluid 53 in the form of culture solution 5 and the third fluid 54 in the form of air 9.

FIGS. 13a, 13b and 13c illustrate three alternative arrangements of the device 51 where the like parts are denoted with like reference numerals. In these arrangements the device 51 further includes a transparent auxiliary film 55 on one or both sides of the barrier 6 so as to contain an additional fluid 56, such as, for example a thin layer of water. In this form the auxiliary film 55 and additional fluid 56 have a relatively minor effect on the optical principles as discussed in relation to FIG. 12. Accordingly, light in the column of water 3 which is incident of the barrier structure 6 still undergoes total internal reflection.

More particularly, FIG. 13a illustrates one possible design where the device 51 further includes a set of transparent plastic bags 57 and 58 whereby sides of the plastic bags 57 and/or 58 form the auxiliary films 55. The column of water 3 is contained in one set of plastic bags 57 and the culture solution 5 is contained in another set of plastic bags 58. The two sets of bags 57, 58 are separated by the barrier 6 with the additional fluid 56 filling the spaces between the barrier 6 and the sides of the plastic bags 57 and/or 58. It is envisaged that the additional fluid 56 may be a very thin layer of water. The air gap 9 in the barrier 6 traps the light by total internal reflection and the auxiliary films have additional fluid 56 on each side so there is little reflection from them. The advantage of this design is that both the column of water 3 and the algae column 5 can be sealed. The disadvantage is that it uses twice as much material. Additionally, it is envisaged that the plastic bags 58 containing the culture solution 5 could include a wavelength shifting element 34.

FIG. 13b illustrates another design in which column of water 3 is contained by the bi-layer barrier 6 and the algae column 5 contained by one of the plastic bags 57 with a thin layer of the additional fluid 56 between the two.

FIG. 13c illustrates another design in which one can contain the column of water 3 using one of the plastic bags 58 and the algae column 5 can be contained by the bi-layer barrier 6 with a thin layer of the additional fluid 56 between the two.

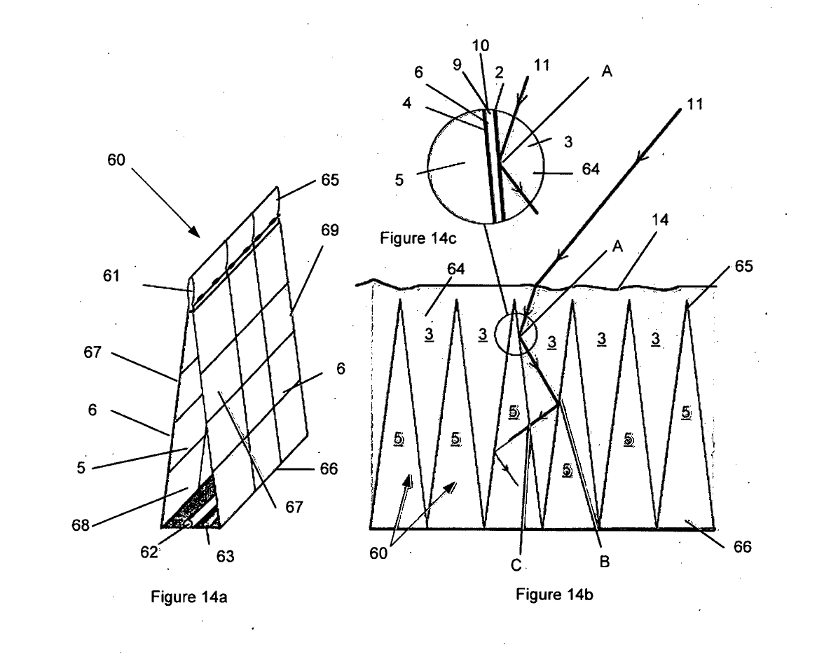

FIGS. 14a to 14c illustrate a further example of a culture apparatus 40. In this example, the culture apparatus 40 includes generally triangularly shaped algae cells 60 located side-by-side within a water medium 64 with columns of water 3 between adjacent algae cells 60.

More specifically, FIG. 14a illustrates a perspective sectional view of one of the algae cells 60. The algae cell 60 is provided in the form of a triangular prism with a front end 68 spaced apart from the back end 69 and main sides 67 which diverge from one another from a tip or top end 65 towards a base or bottom end 66. Each of the main sides 67 include a barrier structure 6 as is further described in relation to FIG. 14c. As may be appreciated from the Figures, the algae cell 60 is triangular in shaped when viewed from one of the front end 68 or the back end 69.

The top end 65 of the algae cell 60 includes an oxygen removal and flotation tube 61 which is buoyant to assist to maintain the orientation of the algae cell 60 in the water medium 64 as well as control of the level of oxygen within the algae cell 60 to maintain favourable growth conditions for algae within the culture solution 5. The bottom end 66 of the algae cell 60 includes a carbon dioxide pipe 62 and fertiliser pipe 63 to respectively supply carbon dioxide and fertiliser to algae within the culture solution 5. The bottom end 66 may also include structural features (not shown) so as be capable of maintaining the shape of the bottom end 66 and weights and/or tethers to another structure (not shown) to assist in maintaining the orientation of the algae cells 60 in the water medium 64.

Referring to FIG. 14b there is illustrated a sectional view of the culture apparatus 40 with a plurality of the algae cells 60 within the water medium 64. The oxygen removal and flotation tube 61, the carbon dioxide pipe 62 and the fertiliser pipe 63 have been omitted for clarity. The plurality of the algae cells 60 are arranged in a side-by-side configuration with the bottom ends 66 of adjacent cells 60 adjoining or being spaced in relatively close proximity to one another. The top ends 65 of the algae cells 60 are preferably located beneath the surface 14 of the water medium 64.

As may be appreciated from FIG. 14b when the algae cells 60 are arranged side-by-side in the water medium 64 the algae cells 60, more particularly the sides 67 of adjacent algae cells 60, define columns of water 3 between adjacent algae cells 60. Each of the defined columns of water 3 is generally inversely triangularly shape relative to the triangularly shaped algae cells 60. Accordingly, as these columns of water 3 are generally flanked by the barrier structures 6 the columns of water 3 each function as a light guide device 1 as is illustrated by light ray 11.

Furthermore, due to the inverse triangular shape of the columns of water 3, as the light ray 11 is reflected down into the column of water 3 the angle of incidence becomes less at each reflection until the incident angle of the light is less than the critical angle and the light passes through the light barrier. More particularly, as may be appreciated from FIG. 14b, the light ray 11 first strikes the barrier structure 6 at location A at an incidence angle greater than the critical angle and is reflected to location B. At location B the incidence angle is still greater than the critical angle therefore the light ray 11 is reflected to location C. At location C the incidence angle is less than the critical angle and therefore the light ray 11 passes through the barrier structure 6 and into the culture solution 5 contained within the algae cell 60.

Referring to FIG. 14c, the barrier structure 6 of the algae cells 60 includes a first wall 2 and a second wall 4 which are spaced apart to define a gap 10 which is generally filled with air 9. The first wall 2 and the second wall 4 are made of a light transmissive polymer material which may preferably be high density polyethylene. Similarly to the other examples disclosed herein, the first wall 2 and/or the second wall 4 may include a wave length shifting element 34. Furthermore, as is shown in FIG. 14c, the barrier structure 6 being located between the water column 3 and the algae solution 5 functions to totally internally reflect light which is incident the barrier structure 6 at an angle greater than the critical angle.

In one form, the algae cells 60 may be deployed in a 1.1 m deep pool which provides the water medium 64. In this instance, the height or distance between the top end 65 and bottom end 66 of the triangular algae cell 60 will be approximately 1.0 m with a bottom end 66 or base width of 0.10 m. Furthermore, although the FIGS. 14a and 14b illustrate sections of the triangular algae cell 60 it should be appreciated that these cells may be configured to be over 100 meters long, that is, there may be 100 m between the front end 68 and the back end 70. Moreover, it should be appreciated that a plurality of algae cells 60 may be utilised in any suitable medium such as a man made pool filled with a fluid such as water or a naturally occurring river, lake, estuary or oceanic environment.

It should be appreciated that whilst triangularly shaped algae cells 60 are described hereinbefore, the algae cells 60 may also be of other shapes which provide barrier structures 6 flanking the columns of water 3 so as to provide a light guide device 1. For example, the main sides of the algae cells 60 could be inwardly curved when viewed from one of the first end or the second end to define columns of water 3 which are parabolic in shape. Alternatively, the main sides of the algae cells 60 could be outwardly curved when viewed from one of the front end 68 or the back end 69 to define columns of water 3 which are parabolic in shape. In yet another form, the main sides 67 of a lower portion of the algae cells 60 may be substantially parallel to one another and the main sides 67 of an upper portion of the algae cells 60 may converge towards another to a tip or top 65. As such, in this form, the algae cells 60 when viewed from one of the front end 68 or the back end 69 have a generally rectangular lower portion located away from the surface 14 of the water medium 64 and a triangular upper portion located towards the surface 14 of the water medium 64.

Throughout this specification the reference to algae is simply by way of example and any reference to algae is intended to include any photosynthetic microorganism. Additionally, the reference to culture solution is intended to include any solution in which a photosynthetic microorganism may be grown.

Many modifications will be apparent to those skilled in the art without departing from the scope of the present invention.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### LIST OF PARTS

1. Light guide device
2. First wall
3. Column of water
3a. Light transmissive medium
4. Second wall
5. Culture solution
6. Barrier structure
7. Gap portion
8. Joined portion
9. Air
9a. Material
10. Gap
11. Incident light
14. Surface
15. Vessel
16. Upper end
17. Light receiving end
18. Base
19. Mirror
20. Ballast
21. Attachment points
22. Cell
30. Light scattering particles
31. Scattered Light
32. Light scattering element
33. Surface texture
34. Wavelength shifting element
40. Culture apparatus
41. Opaque top
42. Algae cell
43. Fluid
44. Directing element
45. Channel
46. Rectangular prism
47. Port
48. Pipe
49. Operation fluid
50. Location
51. Device
52. First fluid
53. Second fluid
54. Third fluid
55. Auxiliary film
56. Additional fluid
57. Plastic bag containing water
58. Plastic bag containing culture solution
60. Triangular algae cell
61. Oxygen removal and floatation tube
62. Carbon dioxide tube
63. Fertiliser tube
64. Water medium
65. Top end
66. Bottom end
67. Main sides
68. Front end
69. Back end

1-28. (canceled)

29. A light guide device for transmitting light including a barrier structure with first and second walls for separating a column of light transmitting medium and a culture solution; wherein the first and second walls are separated by a gap; and wherein the first and second walls are light transmissive to allow light to be extracted from the medium, into the culture solution.

30. The light guide device of claim 29, wherein the light transmitting medium is liquid and the gap is filled with a gas to allow light incident on the barrier structure, within the column of liquid, to be transmitted through the column of liquid by total internal reflection.

31. The light guide of claim 29, wherein the first and second walls are formed of thin flexible material.

32. The light guide device of claim 31, wherein the first and second walls are composed of polymer sheets.

33. The light guide device of claim 31, wherein portions of the first and second walls are joined so as to form joined portions.

34. The light guide device of claim 33, wherein the gap is formed of multiple gap portions defined between joined portions of the walls.

35. The light guide device of claim 29, wherein the minimum width of the gap is 1 micrometer.

36. The light guide device of claim 29, further including a wavelength shifting element for shifting the wavelength of light incident the device.

37. The light guide device of claim 36, wherein at least one of the first and second walls includes the wavelength shifting element.

38. The light guide device of claim 36, wherein the wavelength shifting element is provided in the form of a sheet material located within the culture solution.

39. The light guide device of claim 36, wherein the wavelength shifting element includes fluorescent or phosphorescent materials.

40. The light guide device of claim 29, wherein the device is in the form of a cell and the barrier structure is configured to contain either the medium or the culture solution, separated from the adjacent other of the medium or the culture solution.

41. A culture apparatus, including an array of cells formed in accordance with the cell defined in claim 40.

42. The culture apparatus of claim 41, wherein the adjacent cells share a common barrier structure.

43. The culture apparatus of claim 42, wherein the first and second walls of the barrier structure are formed of thin flexible material connected at joined portions that form a mesh or weave pattern across the barrier structure.

44. The culture apparatus of claim 43, wherein covers are provided over the culture solution and are configured to direct light from over the culture solution into the light guide device.

45. A culture apparatus, including a cell formed in accordance with the cell defined in claim 40, wherein the cell has opposing side walls including the barrier structure, the side walls diverging from one another from a first end of the cell towards a second end of the cell.

46. The culture apparatus of claim 45, wherein there are at least two said cells within the medium, the cells being arranged side-by-side with a column of the medium therebetween, the first end of each cell being located towards a surface of the medium and the second end of each cell being located away from the surface of the medium.

47. The culture apparatus of claim 45, wherein the or each cell is substantially triangular in shape when viewed from one of the front end and the back end of the cell.

48. The culture apparatus of claim 45, wherein the or each cell contains the culture solution and the medium is water.
